(19) United States
(12) Patent Application Publication
(10) Pub. No.: US 2016/0213354 A1
(43) Pub. Date: Jul. 28, 2016

(54) Temperature Measuring Device

(71) Applicant: YONO HEALTH INC., Sunnyvale, CA (US)

(72) Inventors: Peleg Levin, Stanford, CA (US); Zehui Xi, Sunnyvale, CA (US)

(21) Appl. No.: 14/953,301

(22) PCT Filed: Sep. 11, 2015
(86) PCT No.: PCT/US15/00099

§ 371 Date: Nov. 28, 2015

Related U.S. Application Data
(60) Provisional application No. 62/049,890, filed on Sep. 12, 2014.

(51) Int. Cl. A61B 10/00; A61B 5/01; A61B 5/00

(52) U.S. Cl. CPC A61B 10/0012; A61B 5/6817; A61B 5/0008; A61B 5/7275; A61B 5/01; A61B 2010/0019; A61B 2562/0271; A61B 2562/18; A61B 2560/0475

(57) ABSTRACT

An ovulation prediction system comprises an ear temperature measuring device that is configured to continuously measure a person's basal body temperature. In one embodiment, the measured temperature is used to record the person's temperature oscillations during her sleep and use the measured oscillations to predict a woman's ovulation time by comparing it with previously recorded measurements. In particular, the temperature oscillations may be match to a person's sleeping cycle, e.g., marking the beginning and end of the resting time, to increase the likelihood of correctly predicting the ovulation time. To facilitate the analysis of the temperature measurement and allow customization of the ovulation prediction, one embodiment of the system is configured to transmit the measured data wirelessly to a user computer that runs an ovulation prediction algorithm.

This Patent Application is a Non-provisional Application and claims the Priority Date of Application of a co-pending Provisional Application with a Ser. No. 62/049,890 filed by a common Inventor of this Application on Sep. 12, 2014. The disclosures made in Application 62/049,890 are hereby incorporated by reference.

### FIELD OF THE INVENTION

This invention relates to a device for monitoring a patient's basal body temperature. The device can also be applied to monitor in-patients body temperature, heart rate, and other vital signs to save the effort of manual monitoring and data collection. The monitoring can be used for predicting ovulation, measuring sleep quality, or other suitable activity or purpose. In particular, it relates to a device that can be positioned inside a patient's ear for measuring the patient's temperature, sleeping quality, or other vital signs, and can wirelessly transmit these measurements to a smartphone or any other external receiving computing device.

### BACKGROUND

Measuring basal body temperature has been recognized as a way of determining a woman's time of ovulation during her fertility cycle. Timing a woman's ovulation is important for an increased likelihood of conception. Generally, the temperature of a patient's blood circulation in the brain is often indicative of the patient's general physiological state and health. Charting basal body temperature, the lowest temperature the body reaches during a resting period, is a well-known and widely used method of predicting ovulation. To obtain the basal body temperature, a person's temperature is typically measured shortly after the person has awakened and before she engages in any physical activity. However, the most accurate results are obtained when the temperature is measured continuously during the resting state. In particular, at the time of ovulation, a woman only experiences an increase of basal body temperature of about a quarter to 0.3 degree Celsius (about one-half degree Fahrenheit). Unless measured right around the temperature minimum, this slight increase may not be detected due to larger variations in the ambient background temperature.

To eliminate inconsistent basal body temperature readings, a woman typically measures her temperature daily at the same time and under the same conditions. However, even compliance to a strict schedule does not assure an accurate reading of the basal body temperature. This is because physiological events in the woman's body do not necessarily coincide with the time of when she goes to bed or wakes up. In addition, a single temperature measurement per day might not yield enough information to accurately determine the time of ovulation, since other causes, e.g., temporary insomnia, might be the origin of a sudden increase in temperature. Measuring a woman's other physiological functions, e.g., pulse and heart rate, cervical mucus, breast tenderness, in combination with data about her temperature during resting time, can improve the accuracy in predicting her ovulation time.

Traditional thermometers are not well-suited for continuously measuring a person's temperature over or extended resting period. These thermometers lack the capability to measure other physiological functions. In addition, these thermometers are often invasive, e.g. rectal probe, require sterilization, are inconvenient to operate for longer periods, and are limited in their accuracy that is not high enough for determining an increase in temperature when a woman ovulates. For example, a thermometer measuring a patient's skin temperature often has accuracy, since ambient temperature can readily alter its readings. Furthermore, traditional thermometers are not equipped to continuously record temperature date and analyze this data in real-time. For this reason, basal body temperature readings are still recorded by hand and later inputted into computer software for further analysis to predict ovulation. Besides the inconvenience factor, this procedure increases the risk of introducing errors into the prediction by, e.g., inputting an incorrect value into the program. An erroneous reading for just one day may yield unusable results for the entire cycle. Since even slight errors in measuring the basal body temperature would result in a wrong prediction, it is critical that the temperature measurements minimize the error every day. Thus, there is a need for an easy, accurate, and comfortable way of providing continuous temperature monitoring.

During sleep, the human body goes through several sleep cycles where in each cycle an individual goes through different consciousness levels which are categorized as rapid eye movement (REM) and non-rapid eye movement (NREM). The REM type of sleep is associated with the capability of dreaming. While in the NREM type of sleep there is relatively very little dreaming. The sequence in a typical sleep cycle consists of different stages in the NREM sleep (Stages N1-N3, as categorized by the American Academy of Sleep Quality) followed by REM sleep. Published studies have shown that the number and duration of these NREM-REM cycle s varies, with an average of 4 cycles for 8 hours of sleep. The average duration of each of these cycles varies with approximately 70-100 minutes for the first cycle, and 90-120 minutes for the second and later cycles. Individuals who suffer from sleep disorders (e.g., insomnia sleep apnea, restless legs syndrome, narcolepsy) would not experience these sleep cycles sully, and thus could be prone to various health problems. It has also been established by studies that the timing of waking up is also important. As stage N3 in NREM sleep, and REM sleep, are the deepest sleep stages, a person who is awaken during these stages would often carry a feeling of drowsiness during the day. Thus, a feeling of better sleep quality could be obtained by timing the awakening of a person to be at the shallower stages of the sleep, e.g., NREM N1 and N2.

As the, body goes through the NREM-REM sleep cycle, the body temperature is also expected to oscillate with very small amplitude of several tenths of degrees Celsius or less. As these cycles relate to the body temperature fluctuations, the number of temperature fluctuation cycles, their duration, and their amplitude, could provide valuable information about the sleep cycle.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIGS. 1A-1B show diagrams illustrating how a user may use an ear temperature measuring device within a computer network environment, in accordance with some embodiments.

FIGS. 2A-2D illustrate cross-sectional diagrams of a user's ear and an ear temperature measuring device when placed within the ear, in accordance with some embodiments.

FIG. 2E illustrates a cross-sectional diagrams of the ear temperature measuring device when not placed within the ear, in accordance with an embodiment.

FIG. 3 illustrates, a block diagram of an ear temperature measuring device, in accordance with an embodiment.

FIG. 4 illustrates a flowchart of the method for monitoring a patient's basal body temperature with an ear temperature measuring device, in accordance with one embodiment.

FIGS. 4A-4C illustrate half cross-sectional diagrams simulating heat sensitivity of an ear temperature measuring device comprising layers of different thermal conductivity and an enclosing layer at given ambient and ear temperatures.

FIGS. 5A-5C illustrate half cross-sectional diagrams simulating heat sensitivity of an ear temperature measuring device comprising none, one or a dual metal sheath at given ambient and ear temperatures.

FIGS. 6A-6F illustrate half cross-sectional diagrams simulating heat sensitivity of an ear temperature measuring device comprising different geometries and layers of different thermal conductivity at given ambient and ear temperatures.

FIGS. 7A-7B illustrate temperature profile measurements using an ear temperature measuring device, in accordance with some embodiments.

FIG. 8 depicts a typical human sleep process comprising several cycles of NREM-REM stages.

### DETAILED DESCRIPTION

In the following description, numerous specific details are set forth regarding the systems, methods and media of the disclosed subject matter and the environment in which such systems, methods and media may operate, etc., in order to provide a thorough understanding of the disclosed subject matter. It will be apparent to one skilled in the art, however, that the disclosed subject matter may be practiced without such specific details, and that certain features, which are well known in the art, are not described in detail in order to avoid complication of the disclosed subject matter. In addition, it will be understood that the examples provided below are exemplary, and that it is contemplated that there are other systems, methods and media that are within the scope of the disclosed subject matter.

### Ovulation Prediction System

An ovulation prediction system comprises an ear temperature measuring device that is configured to continuously measure a person's body temperature. The Basal Body Temperature (BBT) is obtained from the measurements on a daily basis (or other periodic basis) by getting the lowest body temperature measured continuously over the night. The measured temperature may be used to record the person's temperature variations (oscillations) during her sleep and use the measured oscillations to predict a woman's ovulation time by comparing it with previously recorded measurements. In particular, the temperature oscillations may be match to a person's sleeping cycle, e.g., marking the beginning and end of the resting time, to increase the likelihood of correctly predicting the ovulation time. By measuring the temperature within the person's ear the measurements are more accurate and less affected by ambient temperature changes, thus more precisely reflecting a person's body core temperature. To facilitate the analysis of the temperature measurement and allow customization of the ovulation prediction, the data can be transmitted to a user computer. The data transmission can be performed via a cable, wirelessly, using Bluetooth, or any other suitable transmission means. The user computer can include any suitable computing device including for example, a desktop computer, a laptop computer, a tablet computer, a smartphone, or a dedicated computing device.

In this embodiment, the user computer executes a software program that receives the measured temperature data and provides an interface to the user for inputting additional parameters for employing an ovulation prediction algorithm. In addition, the software program provides a graphical user interface for displaying the recorded measurements and the predicted ovulation cycle to the user. By integrating the ear temperature measuring device with a user computer, the software may provide the user with real-time updates, allowing for a physician to more readily review the measured data, and for automatic data storage.

In another embodiment, the system comprises additional physiological sensors to measure, e.g., a person's heart rate, pulse, respiration, blood pressure, and oxygenation. In some embodiments, the sensors can include an accelerometer or gyroscope to determine a person's movements. Measuring a person's movements may then be used by the software program to identify the start and end time of the person's sleeping cycle and match the corresponding times with the temperature oscillations during this cycle.

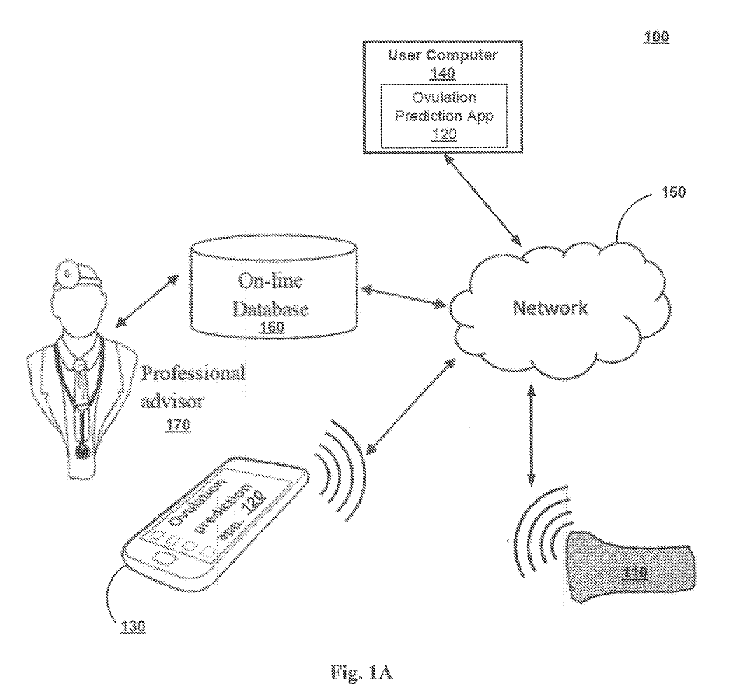

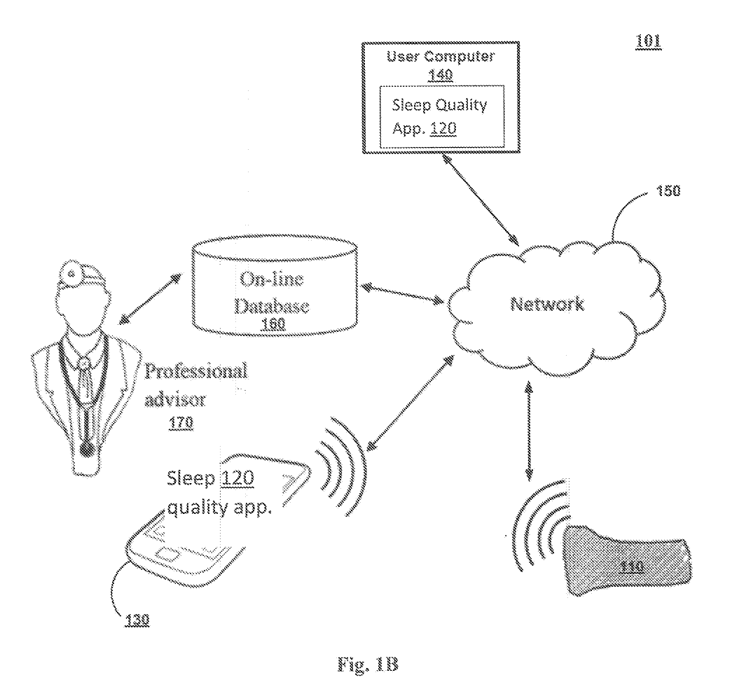

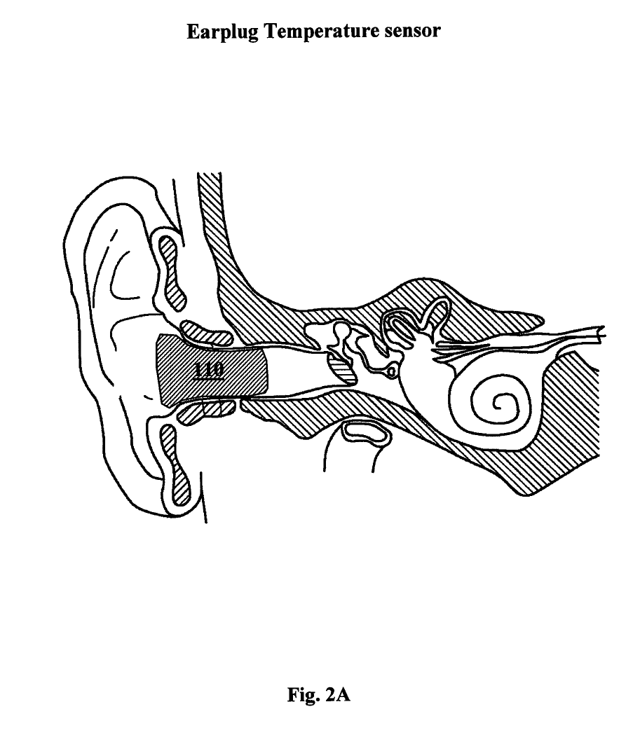

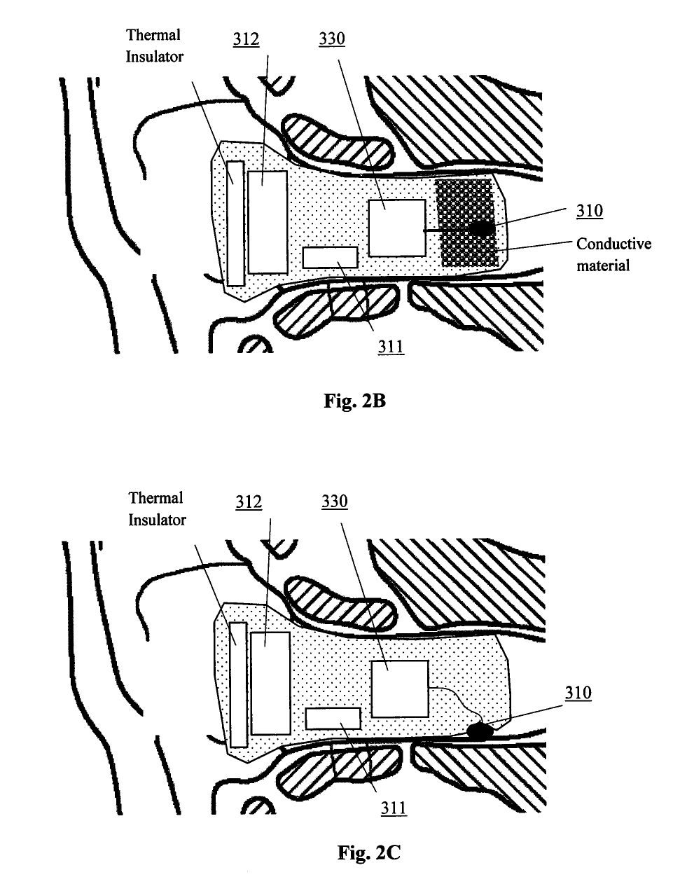

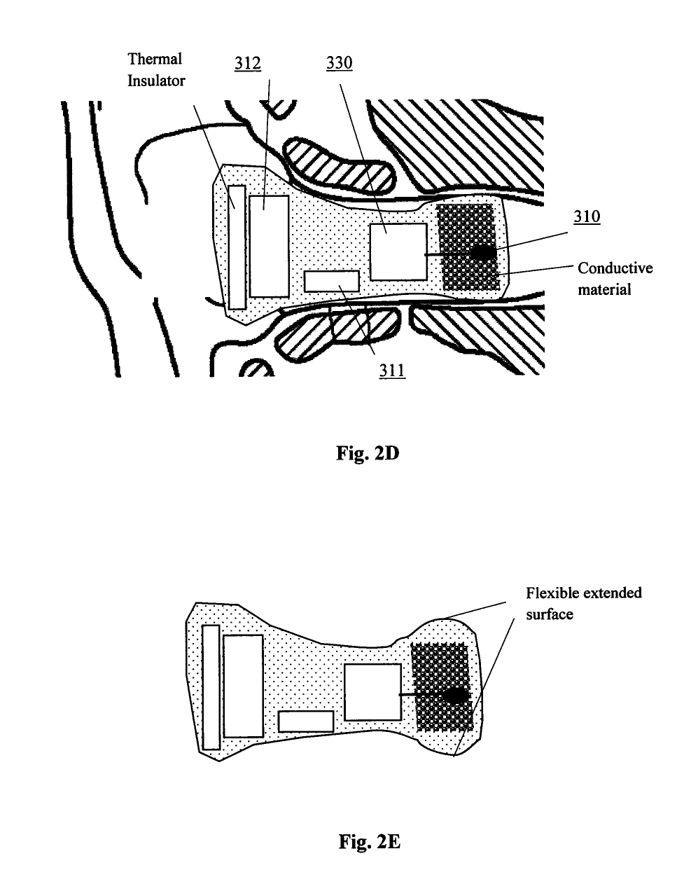

FIG. 1A shows a diagram of an embodiment of an ovulation prediction system 100 that can predict a women's ovulation time by measuring the basal body temperature in the vicinity of the tympanic region of the woman's ear canal. The tympanic region includes the tympanic membrane and the adjacent walls of the ear canal. In one embodiment, the ovulation prediction system 100 comprises an ear temperature measuring device 110, the shape of which closely follows the interior shape of the tympanic region as illustrated in FIGS. 2A-2E. In particular, the ear temperature measuring device 110 can be placed inside the ear to directly contact the surface of the tympanic membrane. FIG. 2C depicts the temperature sensor of the device 110 pressed against the inner skin. In one embodiment, the measuring device 110 comprises a silicon-rubber enclosure to allow for more comfort when wearing the device, thus eliminating the need for any local anesthesia and the risk of damaging the tympanic membrane. In some embodiments, the silicon-rubber enclosure acts as a reliable way of securing the measuring device 110 within the ear canal, preventing its dislodgement when wearing the device over an extended time period or when the patient is sleeping. FIG. 2B shows the structure of an embodiment of the device 110 having extended surface at the tip of the ear plug to assure good pressing against the skin.

Structural embodiments of the ear temperature measuring device 110 include, but are not limited to: (1) the bulk of the device could be made from any type of material (for example, foam/memory foam, silicone, general polymers, thermoplastic, etc.); (2) to reduce heat losses to the surroundings, the device is isolated in the lateral direction (along its axis of insertion into the ear), except at the tip, and is configured to conduct heat from the air cavity in the ear to the temperature sensor; (3) to improve isolation from the ambient, the device might have, near its exterior tip, a region with conductivity below a defined threshold, e.g., existing thermal insulators or a void filled with air with dimensions and structure that precludes internal convection; (4) to assure good contact with the skin, the device 110 is configured to directly pressed against the skin, and/or its structure has a larger diameter at its tip to assure good contact at that region (in this embodiment, the additional structure is made from soft material to assure comfort to the user); (5) the device 110 has a generic fit with different sizes, or custom molded (laboratory made or formed in place) to fit a user's ear and ear canal, and the device 110 being flanged or not flanged, while using different ways of fixture, for example a hook-like structure behind the ear; and (6) device is configured to entirely and/or partially reduce noise and/or external sounds.

In some embodiments, the measuring device 110 is enclosed by biocompatible material, including biocompatible polymers or any other suitable material, to minimize any potential allergic reaction and allow the patient to wear the device for extended time periods. A biocompatible material is either a synthetic or natural material that is not recognized by a body's immune system as a foreign object, thereby evading the immune system's detection and acting as a stealth layer for the measuring device 110. In other embodiments, the outer layer of the ear temperature measuring device is hermetically sealed to prevent water from entering the interior of the device, allowing the device 110 to be washable. Other materials besides silicone rubbers can be used, including, but are not limited to thermoplastics.

Another embodiment of the ear temperature measuring device 110 includes one, two, or more temperature sensing elements or other physiological sensors. A temperature sensor element of 110 includes, but is not limited to: thermistor, thermocouple, thermopile, NTC/PTC, or any other resistance temperature detector (RTD). Alternatively, the temperature sensor element is an infra-red (IR) sensor. To improve accuracy of measurement, an embodiment of the device 110 includes a heating element. The benefit of using a thermistor, instead of an IR sensor, includes that its temperature measurements are not affected by wax buildup inside the user's ear as compared to IR sensors. Embodiments of the ear temperature measuring device 110 can be configured to measure: (1) the body temperature, including BBT, could be made by measuring the temperature of the air inside the ear canal; (2) the temperature of the skin surface in contact with; (3) and/or the temperature of the eardrum.

Other physiological sensors embedded in the ear temperature measuring device 110 include, but are not limited to: (1) an accelerometer and/or gyroscope that senses user movements during the night; (2) pulse oximetry sensor; (3) a brain wave activity sensor (for example, EEG) to measure brain wave activity that directly correlates with sleeping quality; (4) other measuring devices that surrogate signals related to sleeping quality and brain activity, such as sensors for blood pressure, respiration, and oxygenation.

The ear temperature measuring device 110 is also made of conductive material that is used to improve the thermal conductivity between the inner ear skin and the temperature sensor. Conductive material used for the device 110 may include an anisotropic material with radial high conductivity and lateral low conductivity. Furthermore, the material may have special structural characteristics such as windings of small in diameter conductor. In case this material is rigid, e.g., the material being copper, the windings could provide mechanical flexibility while maintaining high conductivity in that region.

The ovulation prediction system 100 further comprises an ovulation prediction software 120 that may run on a smartphone 130 or a user computer 140, all communicatively coupled through a communications network 150 (e.g., the Internet or a wireless network, such as Bluetooth, NFC or WiFi) with the ear temperature measuring device 110. For example, the ear temperature measuring device 110 may be programmed to communicate with the smartphone 130 using a networking protocol such as transmission control protocol/internet protocol (TCP/IP) or any other suitable protocol. Although only one of each type of computing system is shown, in practice many of each type of computing system exist on the Internet, and the various instances of each type of computing systems interact with each other on a frequent basis.

In one embodiment, a user uses a computing device configured to run an ovulation prediction software application 120 to receive the temperature data measured by the ear temperature measuring device 110. For sake of clarity, reference to a user is a reference to the user's computing device, as a mechanism of abstracting away from the actual human actor controlling the computer. Each computing device may include conventional components of a computing device, e.g., a processor, system memory, a hard disk of solid state drive, input devices such as a mouse, a keyboard or touch screen, and/or output devices, such as a monitor or display.

The computing device comprises one or more client devices that can receive user input and can transmit and receive data via the network 150. For example, the client devices may be desktop computers 140, laptop or tablet computers (not shown), smartphones 130, personal digital assistants (PDAs, not shown), or any other device including computing functionality and data communication capabilities. The client devices are configured to communicate via network 150 with the ear temperature measuring device 110, which may comprise any combination of local area and/or wide area networks, using both wired and wireless communication systems. In other embodiments, the ovulation prediction system 100 may include additional, fewer, or different components for various applications. Conventional components such as network interfaces, security mechanisms, load balancers, failover servers, management and network operations consoles, and the like are not shown so as to not obscure the details of the system.

The embodiment in FIG. 1A also includes an online database 160 that stores the data measured by the ear temperature measuring device 110 and the analysis data computed by the client devices 130, 140. In this embodiment, the ovulation prediction systems 120 stores data generated by the ear temperature measuring device 110 in an online database 160 and may communicate data to physician or other medical staff who then may assist the system in correctly predicting the time of ovulation.

FIG. 1B shows a similar diagram with similar operation and functionality as FIG. 1A, but is directed to an embodiment of a sleep quality system 101.

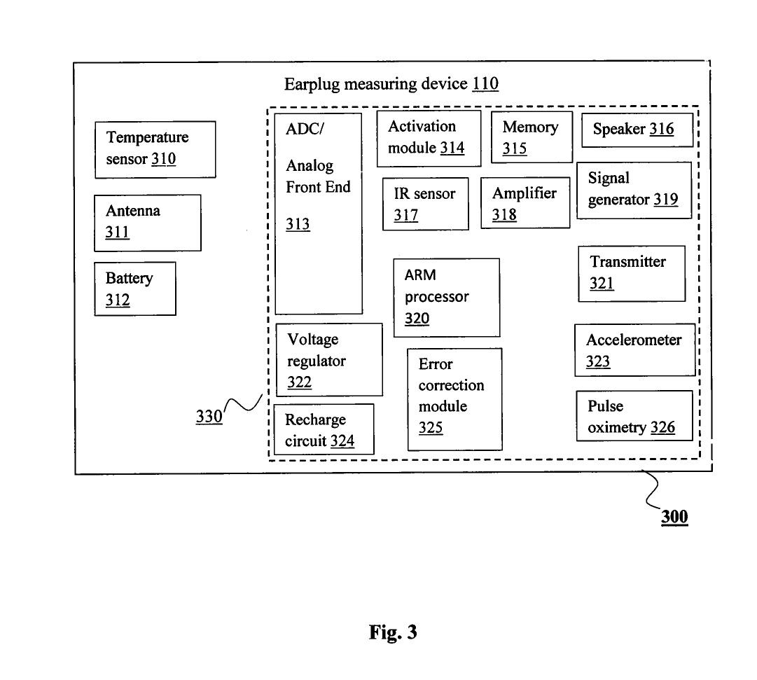

FIGS. 2B to 2D illustrate the structure of the temperature measuring device 110. The temperature measuring device 110 includes a temperature sensor 310 disposed on a conductive material used to improve the thermal conductivity between the inner ear skin and the temperature sensor. Preferably, the conductive material is made of an anisotropic material with radial high conductivity and lateral low conductivity. It could have special structural characteristics such as windings of small in diameter conductor. In case this material is rigid (for example copper), the windings could provide mechanical flexibility while maintaining high conductivity in that region. The temperature sensor 310 transmits the temperature measurements to an input port on a print circuit board (PCB) 330. The PCB 330 includes electronic components as that shown in FIG. 3. The temperature measuring device 110 further includes an antenna 311 for transmitting signals of the temperature measurements. The temperature measuring device 110 further includes a battery 312 for providing power to operate the temperature measurement function and a thermal insulator to thermally insulate the temperature measuring device from the ambient temperature outside of the ear.

General structure of the temperature measuring device implemented as an ear plug:

a. The bulk of the ear plug could be made from any type of material (for example, foam/memory foam, silicone, general polymers, thermoplastic etc.)

b. Preferably, to reduce heat losses to the surroundings, the ear plug is mainly isolating in the lateral direction (along its axis of insertion into the ear), except at the tip, when it's necessary to conduct heat from the air cavity in the ear to the temperature sensor.

c. The ear plug could have a generic fit with different sizes, or custom molded (laboratory made or formed in place) to fit one's ear and ear canal. The ear plug could be made flanged or not flanged, and could use any type of fixture (for example a hook-like structure behind the ear).

d. The ear plug could be structurally designed to entirely/partially reduce or not reduce noise and/or external sounds.

Additional biometric components can be implemented on the PCB 339:

e. An accelerometer and/or gyroscope might be included to sense user movements during the night.

f. Pulse oximetry sensor.

g. Possibly might include a brain wave activity sensor (for example, EEG). Brain wave activity correlates directly with sleeping quality.

h. Measuring other surrogate signals that relate to sleeping quality/brain activity (for example, sensors for blood pressure, respiration, oxygenation, etc.).

### Configuration of the Ear Temperature Measuring Device

The diagram in FIG. 3 shows components of the ear temperature measuring device 110 according to one embodiment. In this embodiment, the device 110 includes an activation module 314, a sensor 310, and an error correction module 325. The device 110 may also include a processor, a system memory and a power supply, and may be configured to generate a data signal by the signal generator 319 to be transmitted via the communication network 150 to the user's smartphone or computer. In alternative configurations, different and/or additional modules can be included in the device 110. Other electronic components may include: an ARM CPU (or other CPUs, micro controllers); an analog to digital converter (ADC); an analog front end chip (AFE); an voltage divider; non-volatile memory to store measurements and to allow asynchronous reads via RF data transmission; a rechargeable or non-rechargeable battery; a voltage regulator circuit; a RF transmitter, receiver and antenna, e.g., Bluetooth, Wi-Fi and NFC; a battery recharging circuit configured for wired or wireless recharging; a miniature speaker for providing feedback to the user potentially used in combination with a Smart Alarm feature; and/or any other suitable component or combination of components. A Smart Alarm feature may be based on the monitored body temperature profile, and could be activated to awake the user at an optimal times (for example, possibly at the high peaks of the temperature which could be related to more conscious stages of the user). The alarm could be active through a miniature speaker inside the device, or it can trigger an external alarm such as, for example, a smart phone.

### Activation of Ear Temperature Measuring Device

In one embodiment, the activation module 314 of the ear temperature measuring device 110 continuously monitors the temperature. Once the device 110 inserted into an ear, the activation module 314 senses an increase in the measured temperature that exceeds a user-specified threshold level with the measured value approximating the body temperature. This threshold level can be set to a value significantly higher than the ambient temperature of storage of the device, which is usually below 35° Celsius. The threshold level could be set at 35.5-36.0 Celsius, or any other suitable Celsius or Celsius range, which is within the spectrum of the normal human body temperature. Once module 314 senses the threshold level, the device starts by first transmitting the stored measurements to the receiver such as a computer or smartphone, or directly goes into taking measurements mode. Alternatively, once the device senses a decrease from the level of measured basal body temperature, a decrease which might be as low as ˜1° Celsius or any other suitable temperatures, and thus happen relatively fast after removing the device from the ear, the device is triggered to transmit the collected data to the said receiver, and is triggered back to stand-by mode. If the data was transferred in prior stage, the device goes to stand-by mode directly.

In another embodiment, the device triggering is made by sensing the capacitance and/or resistance of its surroundings. In case resistance is measured, the device includes a couple of electrical contacts to close a circuit through the skin.

In yet another embodiment, if the device is coupled with a sensor for monitoring EEG signals, the device could be operated in the some mode as described in Paragraph [0034], only here the device triggering is made by sensing the EEG signal.

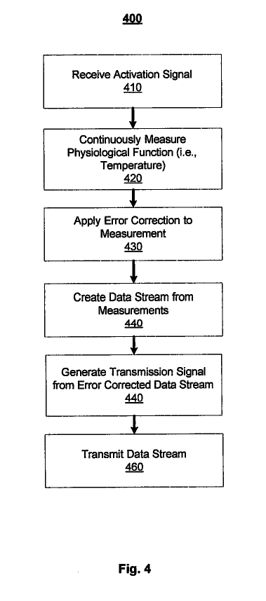

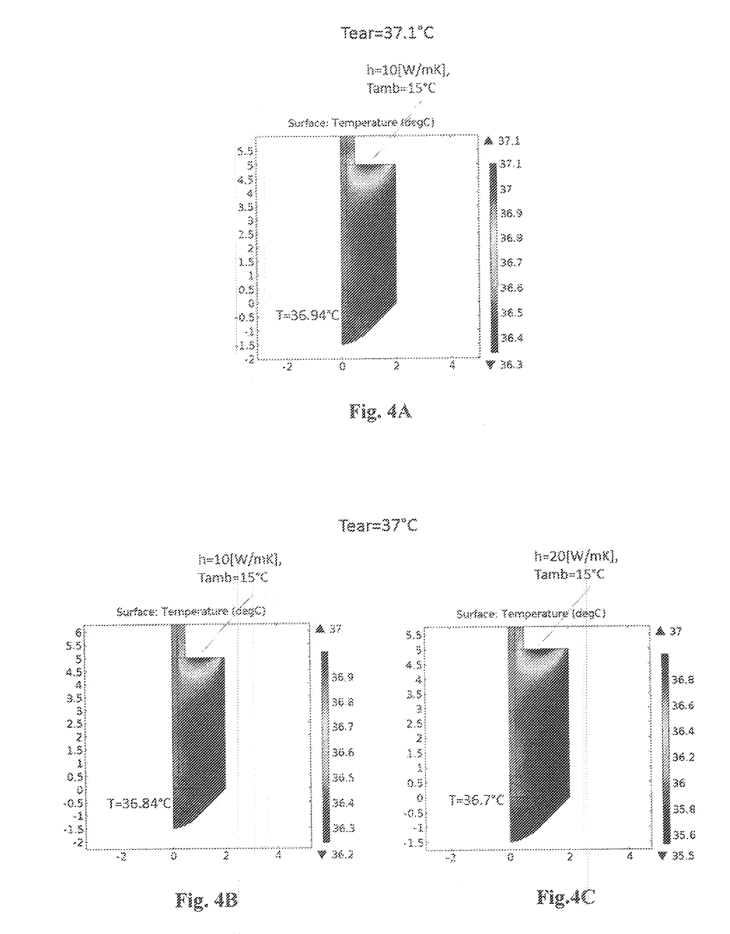

The flowchart in FIG. 4 illustrates a method for monitoring a patient's basal body temperature with an ear temperature measuring device 110, in accordance with one embodiment. The first step in monitoring the basal body temperature comprises activating of the device 110 by the activation module 314.

### Temperature Sensor

The sensor 310 of the ear measuring device 110 comprises a thermistor or temperature transducer. In one embodiment, the sensor 310 comprises one or more thermistors.

### Physiological Sensors

In one embodiment of FIG. 3 the ear temperature measuring device 110 comprises multiple sensors that can measure a person's physiological functions. These sensors include but are not limited to physiological sensors to measure, e.g., a person's heart rate, pulse, respiration, blood pressure, and oxygenation. In some embodiments, the sensors comprise an accelerometer or gyroscope to determine a person's movements.

FIGS. 4A-4C illustrate half cross-sectional diagrams when simulating heat sensitivity of an ear temperature measuring device that comprised layers of different thermal conductivity and an enclosing layer of silicon rubber at a given ambient and ear temperature.

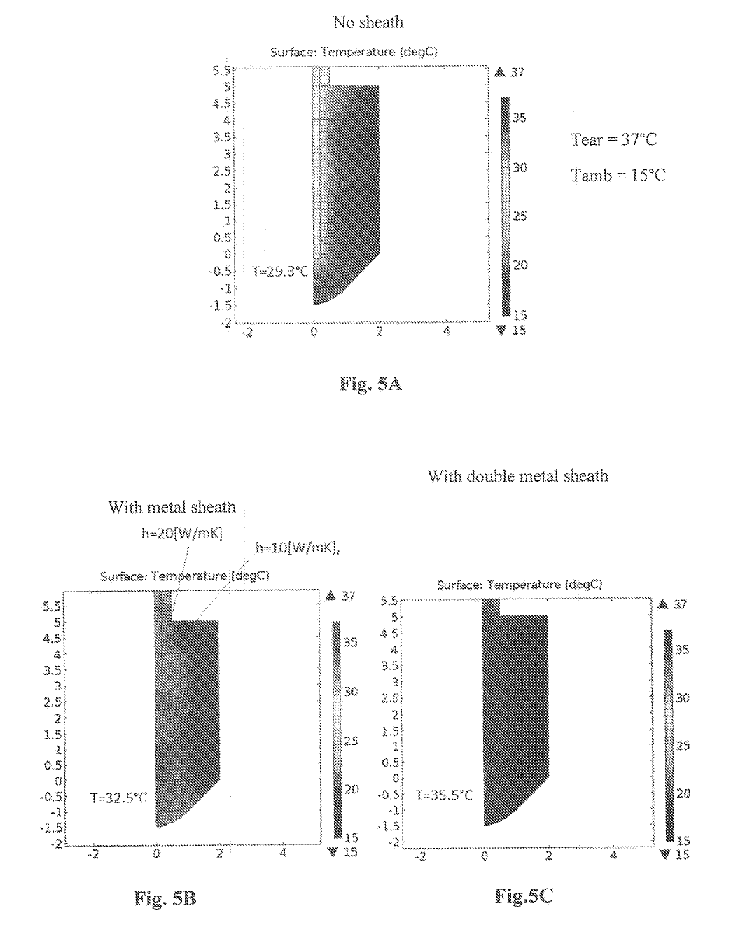

FIGS. 5A-5C illustrate half cross-sectional diagrams simulating heat sensitivity of an ear temperature measuring device that comprised no, one or a dual metal sheath at a given ambient and ear temperature.

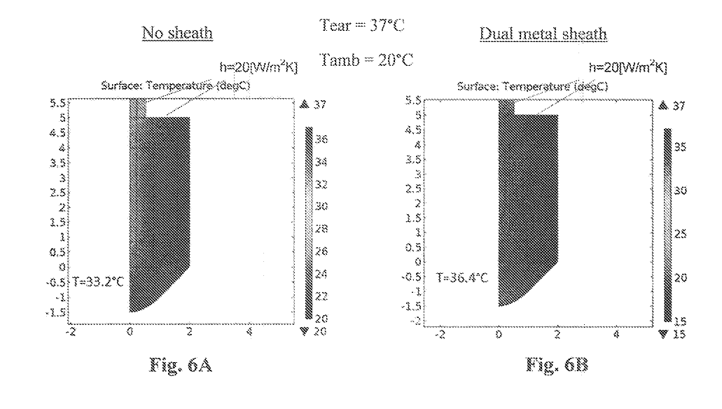

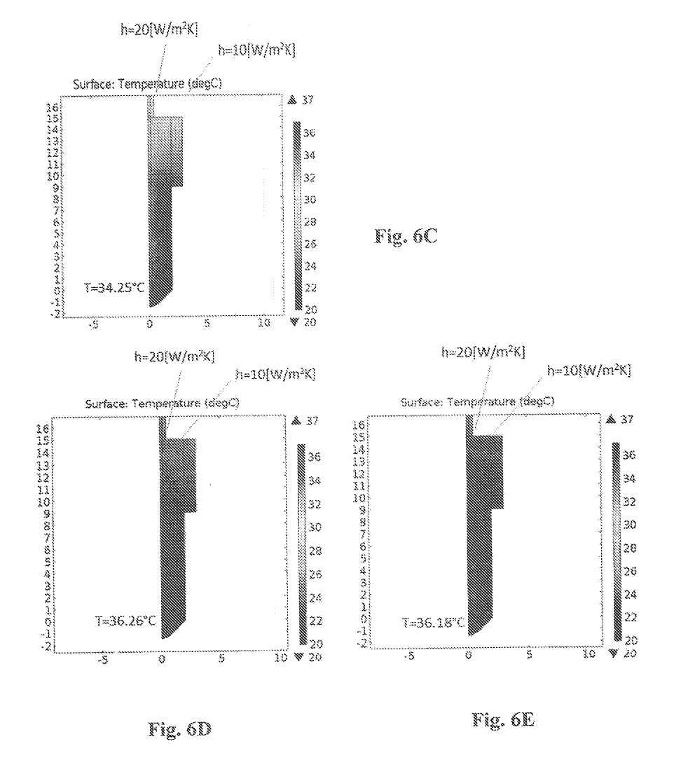

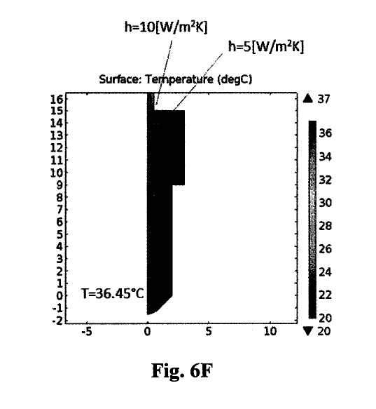

FIG. 6A-6F illustrate half cross-sectional diagrams simulating heat sensitivity of an ear temperature measuring device that comprised different geometries and layers of thermal conductivity at a given ambient and ear temperature.
1. Influence of ambient conditions: FIGS. 4A-C demonstrate the sensitivity of the measurement on the BBT temperature change. A rise of 0.1° Celsius in the BBT results in the same rise of the measured temperature. FIGS. 4B-C demonstrate the sensitivity of the measurement on increased air flow over the ear of the user. A rise in heat convection coefficient of 10 W/mK resulted in 0.14° Celsius shift in the measured temperature.
2. Influence of the addition of a highly conductive material/thermal mass (e.g., metal sheath): FIGS. 5A-C demonstrate that, for the case where a thin wire extends away from the device into the ambient, the addition of a highly conductive material greatly improves the accuracy of the measurement and reduces the influence of the ambient conditions on the measurement.
3. Influence of the addition of a highly conductive material/thermal mass (e.g., metal sheath): FIGS. 5A-B demonstrate that, for the case where a thin wire extends away from the device into the ambient, the addition of a highly conductive material greatly improves the accuracy of the measurement and reduces the influence of the ambient conditions on the measurement. The presented case is for the case where the heat convection coefficient form the prototype's wire and exposed end is 20 W/mK and 10 W/mK, accordingly.
4. Influence of the addition of a highly conductive material/thermal mass (e.g., metal sheath)—For a different form factor (actual wired prototype geometry): FIGS. 6A-F demonstrate that, for the case where a thin wire extends away from the device into the ambient, the addition of a highly conductive material greatly improves the accuracy of the measurement and reduces the influence of the ambient conditions on the measurement. The presented case is for several possible ambient conditions, defined by the various heat convection coefficients, and a constant ambient temperature of 20 degrees Celsius.

As concluded from the case which is depicted in FIG. 6E, an optimal gap should be kept between the conductive material/thermal mass and the exposed tip of the plug, bellow which excessive heat loss to the ambient occurs and the measured temperature becomes less accurate,

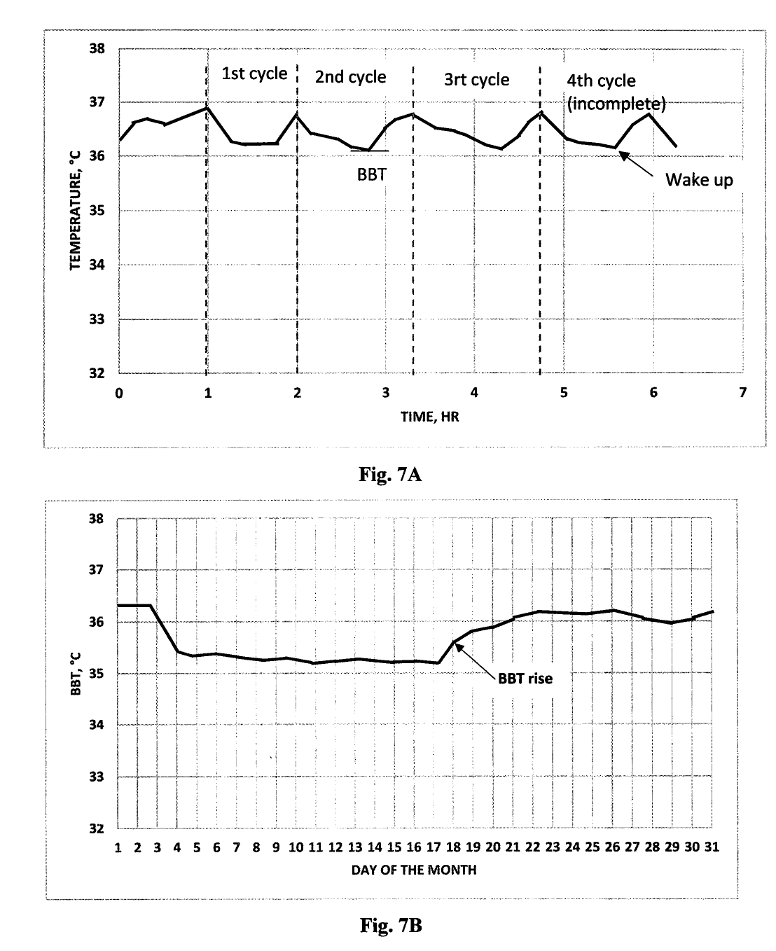

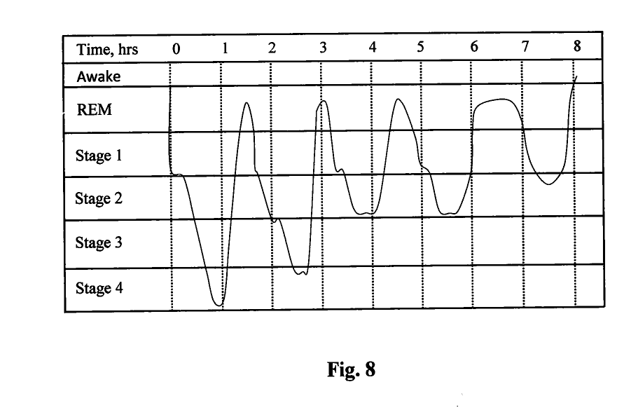

FIGS. 7A-B illustrate examples of temperature measurements using an embodiment of the ear temperature measuring device 110.

In some embodiments, the ear temperature measuring device 110 is configured to measure the following:

Sample Data
It may be noticed that the temperature fluctuates during sleep, consisting of several cycles.
The lowest reachable temperature is considered as the basal body temperature.
The duration of the different cycles varies, and so is their amplitude.

Measuring Basal Body Temperature (BBT) based on the measured temperature profile, the BBT is obtained on a daily basis:
The form of the BBT profile and its values carry information that relates to ovulation, sleep quality, cancer and thyroid disease.
For ovulation prediction: BBT rises after ovulation by approximately 0.3 to 0.6° Celsius (or even 1.5° Celsius). A woman is assumed to have ovulated after observing 3 consecutive days of temperature elevation. BBT predicts the peak of fertility, helping a couple to plan the optimal time for coitus. The fertile interval ends on the fourth morning after peak day.
The BBT method could be combined with other user-inputs, such as calendar calculations, period, mucus changes, etc.
For contraception: Basal body temperature data can be used with other method such as calendar method, etc. as part of Natural Family Planning. A couple should refrain from coitus during the most fertile days and avoid getting pregnant.
As the accuracy of the BBT monitoring method strongly depends on the quality of sleep, and requires at least 4-6 hours of uninterrupted sleep the preceding night, the quality of sleep itself should also be measured to conclude whether the measurement is reliable.

Measuring Sleep Quality

The following figure depicts a typical, 8 hours long, sleep cycle:
Looking at the figure, the sleeping process consists of several cycles, where in each cycle the user goes through different consciousness levels. As these cycles relate to the body temperature fluctuations, the number of temperature fluctuation cycles, their duration, and their amplitude, could provide valuable information about the sleep cycle.

The captured temperature profile should by itself provide valuable data about the quality of sleep, but it also may be combined with other sensors such as accelerometer, gyroscope, oximetry, pulse, or any other biometric sensor as described in the product description.

Smart Alarm Feature
Based on the monitored body temperature profile, a smart alarm could be activated to awake the user at an optimal time (For example, possibly at the high peaks of the temperature which could be related to more conscious stages of the user). The alarm could be active through a miniature speaker inside the device, or it can trigger an external alarm such as, for example, a smart phone.

Other Disease Diagnosis
The device can also be used by patients with other diseases that could be correlated to BBT and provide diagnosis features. The device and/or method aim at measuring the core body temperature which could be used in the following applications:
   Ovulation identification & prediction/contraception based on the basal body temperature.
   Sleep quality monitoring based on the temperature fluctuations while sleeping.
   Hormonal disorder.
   The later could be also combined with a Smart Alarm feature which is set to waking the user at the optimal timing in the sleep cycle.
   Provide valuable data for Cancer, Hypothyroidism, or Hyperthyroidism patients by comparing their Basal Body Temperature with that of healthy people.

The device operates by any combination of the following functionalities:
Sensing the temperature in the ear canal
   In particular, observing the temperature fluctuations.
Sensing the heart beat rate/pulse
Sensing blood pressure
Sensing other signals

For monitoring sleep quality or other: the temperature fluctuations in the continuously measured temperature profile, possibly including the number of cycles, their duration and amplitude, etc. In case of a value of one or more of the measurements exceeds a set threshold, the system alerts the physician/patient.

Baby Temperature Monitor:

Many parents are having trouble continuously measuring their baby's body temperature since it's very dangerous for a baby to be on fever. When the thermometer is used as a baby temperature monitor, it will continuously send the signals, via cable, Wi-Fi, Bluetooth, or other medium, to the parents' computing devices. This will save the parents' efforts of periodically and continually measuring the baby's body temperature (e.g., every 30 minutes, 1 hour, or any other suitable frequency), especially at night. Its can be dangerous when the thermometer is used on babies without their parents' constant attention since babies may tear off the thermometer, play with the thermometer, or even try to eat the thermometer. Once the baby tears off the thermometer, the sensor on the thermometer will sense that the earbud is taken off and will send a warning to the parents' computing devices, and the parents could go to the baby immediately to handle the issue.

Alternative Applications

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible computer readable storage medium or any type of media suitable for storing electronic instructions, and coupled to a computer system bus. Furthermore, any computing systems referred to in the specification may include a single processor or may be architectures employing multiple processor designs for increased computing capability.

Embodiments of the invention may also relate to a computer data signal embodied in a carrier wave, where the computer data signal includes any embodiment of a computer program product or other data combination described herein. The computer data signal is a product that is presented in a tangible medium or carrier wave and modulated or otherwise encoded in the carrier wave, which is tangible, and transmitted according to any suitable transmission method.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

We claim:

1. An ovulation prediction system comprising:
an ear temperature measuring device configured for plugging into an ear for continuously measuring a basal body temperature (BBT) in a vicinity of a tympanic region of an ear canal; and
the temperature measuring device further comprises a temperature sensor for continuously measuring the BBT in the ear canal.

2. The ovulation prediction system of claim 1 wherein:
the temperature sensor comprises a thermistor.

3. The ovulation prediction system of claim 1 wherein:
the temperature sensor comprises a thermocouple.

4. The ovulation prediction system of claim 1 wherein:
the temperature sensor comprises a thermopile.

5. The ovulation prediction system of claim 1 wherein:
the temperature sensor comprises a resistance temperature detector (RDT).

6. The ovulation prediction system of claim 1 wherein:
the temperature measuring device further comprises a thermal conductive support surrounding and supporting the temperature sensor to improve a thermal conductivity between the inner canal and the temperature sensor.

7. The ovulation prediction system of claim 1 wherein:
the temperature measuring device further comprises a thermal insulator disposed near a backend opposite the temperature sensor to insulate the ear canal from an external ambient temperature.

8. The ovulation prediction system of claim 1 wherein:
the temperature measuring device further comprises a data memory for storing temperature measurements continuously measured by the temperature sensor.

9. The ovulation prediction system of claim 1 further comprising:
a temperature measurement analyzing device; and
the temperature measuring device further comprises an antenna for transmitting the temperature measurements to the temperature measurement analyzing device.

10. The ovulation prediction system of claim 1 wherein:
the temperature measuring device further includes a battery for providing electric power to operate the temperature measuring device.

11. The ovulation prediction system of claim 1 wherein:
the temperature measuring device further includes a physiological sensor for measuring another physiological function.

12. The ovulation prediction system of claim 1 further comprising:
a wireless telecommunication device to communicate and control the temperature measuring device.

13. A method for performing an ovulation prediction comprising:
configuring and plugging an ear temperature measuring device into an ear; and
implementing a temperature sensor in the ear temperature measuring device for continuously measuring a basal body temperature (BBT) in a vicinity of a tympanic region of an ear canal.

14. The method of claim 13 wherein:
the step of implementing the temperature sensor comprises a step of implementing a thermistor in the temperature measuring device.

15. The method of claim 13 wherein:
the step of implementing the temperature sensor comprises a step of implementing a thermocouple in the temperature measuring device.

16. The method of claim 13 wherein:
the step of implementing the temperature sensor comprises a step of implementing a resistance temperature detector (RDT) in the temperature measuring device.

17. The method of claim 13 wherein:
the step of configuring the temperature measuring device further comprises step of surrounding a thermal conductive support around the temperature sensor to improve a thermal conductivity between the inner canal and the temperature sensor.

18. The method of claim 13 wherein:
the step of configuring the temperature measuring device further comprises step of disposing a thermal insulator near a backend opposite the temperature sensor to insulate the ear canal from an external ambient temperature.

19. The method of claim 13 wherein:
the step of configuring the temperature measuring device further comprises step of implementing a data memory in the temperature measuring device for storing temperature measurements continuously measured by the temperature sensor.

20. The method of claim 13 further comprising:
employing a temperature measurement analyzing device to communicate and
receiving the temperature measurements from the temperature measuring device for analyzing and performing the ovulation prediction.

21. The method of claim 13 wherein:
the step of configuring the temperature measuring device further comprises step of implementing a physiological sensor for measuring another physiological function.

22. The method of claim 13 further comprising:
implementing a wireless telecommunication device to communicate and control the temperature measuring device.
